(19) United States
(12) Patent Application Publication
(10) Pub. No.: US 2006/0047208 A1
(43) Pub. Date: Mar. 2, 2006

(54) Apparatus and method for measuring quantity of exercise through film-type pressure sensor

(75) Inventor: Jung-min Yoon, Seoul (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/213,698

(22) Filed: Aug. 30, 2005

(30) Foreign Application Priority Data
Aug. 30, 2004 (KR) 10-2004-0068547

(51) Int. Cl. A61B 5/02

(52) U.S. Cl. 600/500

(57) ABSTRACT

An apparatus and method for measuring exercise quantity through a film-type pressure sensor are provided. The apparatus for measuring exercise quantity through a film-type pressure sensor comprises a film-type pressure sensor to sense pulse pressure of a user's body, a main body to calculate and display a heart rate (HR) and calories consumed based on the HR, and a band that is coupled to the main body and to which the film-type pressure sensor is attached. The band contains a first band portion which is coupled to the main body and a second band portion which contains the film-type pressure sensor and is coupled to the first band portion. The second band portion contains at least one projection to enhance the detection of the pulse pressure of the user.

### CROSS-REFERENCE TO RELATED APPLICATION

This application claims priority from Korean Patent Application No. 10-2004- 0068547 filed on Aug. 30, 2004, in the Korean Intellectual Property Office, the disclosure of which is incorporated herein in its entirety by reference.

### BACKGROUND OF THE INVENTION

1. Field of the Invention

Apparatuses and methods consistent with the present invention relate to exercise measurement using a film-type pressure sensor. More particularly, the present invention relates to an apparatus and a method for measuring the quantity of exercise of a user using a film-type pressure sensor of a wrist watch-type exercise meter capable of easily and conveniently measuring the calories burned while exercising.

2. Description of the Related Art

FIG. 1A illustrates a construction of an exercise meter to measure the quantity of exercise of a user, which employs a conventional electric sensor. An exercise meter may be understood as referring to a device which is worn by a user and which can easily and conveniently measure calories burned during exercise after physical information such as height and weight are inputted therein.

As illustrated, the conventional exercise meter comprises a chest band to which an electric sensor is attached, and a heart rate monitoring watch to monitor the heart rate (heartbeats per minute; hereinafter referred to as “HR”). Measurement by the exercise meter is based on the heartbeats per minute measured by the electric sensor.

The chest band is put on the user's chest and the watch-type exercise meter is put on the user's wrist, and data measured by the chest band is transmitted to the watch-type exercise meter by use of a radio frequency RF.

Then, the user's heart rate is calculated by the exercise meter based on the transmitted data, on which basis the calories burned during exercise are calculated.

To measure a user's heart rate with the use of the electric sensor, the chest band must be put on the user's chest. The two electric sensors mounted on the chest band should be put on both sides of the chest.

To start the measurement, a start button on the watch-type exercise meter is pressed. Next, the two electric sensors on the chest band measure the potential difference between them. Since regular motions of the human heart cause the user's physiological electric potential to change regularly, heart activity can be monitored through measurement of the electric potential. It is for this reason that the potential difference is measured.

Measured changes in the electric potential are transmitted to the exercise meter by use of the RF. The exercise meter calculates the user's HR based on the transmitted data.

Based on the calculated HR, the calories burned while the user is exercising are calculated. It is known that, while a person is working out, his or her HR has a linear relation with the quantity of oxygen consumed, and the quantity of oxygen consumed has a linear relation with the calories oxidized in the body; thus, the consumed calories can be calculated using these relationships.

Subsequently, the HR is measured at regular predetermined intervals, and the calories burned during exercise can be calculated on this basis.

However, measurement of the HR using the electric sensor requires that electrodes be in constant contact with the user's skin since the HR is calculated using them, and thus, the chest band should be tightly attached to the user's chest. This tightly attached chest band causes discomfort to users, and users have been reluctant to use the chest band for this reason. Also, data may be distorted during transmission because of interference.

FIG. 1B illustrates a construction of an exercise meter to measure the quantity of exercise of a user using a conventional accelerometer.

As illustrated, there are two types of accelerometer-based exercise meters to calculate calories burned during exercise: a pager-type and an armband-type. A pager-type exercise meter is put on the user's waist and an armband-type exercise meter is put on the user's arm.

Both pager-type and armband-type exercise meters use an accelerometer sensor to calculate the user's speed, and on this basis the calories burned during exercise are calculated.

Physically, since energy (calories) is proportional to the square of speed, the consumed calories can be calculated by obtaining the speed of the runner. The acceleration is measured in order to calculate the consumed calories.

A user first wears either of the exercise meters in an appropriate way (e.g., a pager-like device on the user's waist or an armband-like device on the user's arm).

When the user begins exercising, three accelerometer sensors of the exercise meter in fixed positions measure the X, Y and Z accelerations. Based on the measured accelerations, the speed is calculated and the calories consumed burned during exercise are calculated based on this speed.

However, this measurement using the accelerometer sensor is disadvantageous in that it requires that the exercise meter to be put at a fixed position on the body. Respective body parts of a person who is exercising vary in speed and direction; thus when the meter is placed at different positions on a body, the meter will represent different calorie amounts, even when the person does the same exercise. For this reason, the accelerometer sensor should be put at a fixed position on the body.

The calories consumed per unit time have been obtained through experiments, when users exercise while wearing the exercise meters employing the accelerometer sensors, the calories consumed during the whole exercise are calculated on this basis.

Since a user's hands and feet move in different ways, body parts that scarcely move such as the waist and upper arm are preferred positions for the exercise meter.

However, restricting the exercise meter to specific body parts makes it somewhat undesireable. Further, the user has to wear a waist belt in order to put the pager-type exercise meter on his or her waist, which may cause the exercise meter not to work properly while he or she is exercising.

Korean Unexamined Patent Publication No. 2002-080831 entitled “Portable Pulse Examining Device” discloses a film-type semiconductor pressure sensor and a pulse examining device using a low band filter to only filter low frequency pulse signals, wherein the pressure sensor is miniaturized and is attached to a watch band in order to measure the pulse. However, this reference discloses no method for calculating the calories burned during exercise using an exercise meter equipped with a film-type pressure sensor, which is claimed in the present invention.

### SUMMARY OF THE INVENTION

The present invention provides a method for ascertaining the calories consumed during exercise, with the use of a wrist watch-type exercise meter having a film-type pressure sensor while a user is wearing it to exercise.

The present invention also provides an exercise meter whereby a user's heart rate per minute and calories consumed during exercise can be readily measured and ascertained, without giving the user any physical burden or sense of oppression.

According to an aspect of the present invention, there is provided an apparatus for measuring exercise quantity through a film-type pressure sensor, comprising a film-type pressure sensor to sense pulse pressure of a user's body, a main body to calculate and display a HR per minute and calories consumed based on the HR, and a band that is coupled to the main body and to which the film-type pressure sensor is attached.

According to another aspect of the present invention, there is provided a method for measuring exercise quantity using a film-type pressure sensor, comprising measuring the pulse pressure of a user who is exercising, removing noise from the measured pulse pressure signal, calculating the user's HR (per minute), based on the noise-free pulse pressure, calculating the calories consumed by the user based on the calculated HR, and displaying the calculated calories consumed and the HR.

### BRIEF DESCRIPTION OF THE DRAWINGS

The above and other aspects of the present invention will become more apparent by describing in detail exemplary embodiments thereof with reference to the attached drawings in which:

FIG. 1A illustrates a construction of an exercise meter to measure the quantity of exercise by use of a conventional electric sensor;

FIG. 1B illustrates a construction of an exercise meter to measure the quantity of exercise by use of a conventional accelerometer;

FIGS. 2A and 2B illustrate a construction of an apparatus for measuring the quantity of exercise using a film-type pressure sensor according to an exemplary embodiment of the present invention;

FIGS. 3A and 3B illustrate a construction of a film-type pressure sensor and a band in the exercise quantity measuring apparatus according to an exemplary embodiment of the present invention;

FIG. 4A and 4B illustrate a construction of the band of the exercise quantity measuring apparatus using a film-type pressure sensor according to an exemplary embodiment of the present invention;

FIG. 5 is a block diagram illustrating an internal construction of the exercise quantity measuring apparatus using a film-type pressure sensor according to an exemplary embodiment of the present invention;

FIGS. 6A, 6B and 6C illustrate removal of noise using an adaptive filter according to an exemplary embodiment of the present invention;

FIG. 7 is a flow chart illustrating measurement of exercise quantity through a film-type pressure sensor according to an exemplary embodiment of the present invention; and

FIG. 8 is a flow chart illustrating removal of noise in the exercise quantity measuring method through a film-type pressure sensor according to an exemplary embodiment of the present invention.

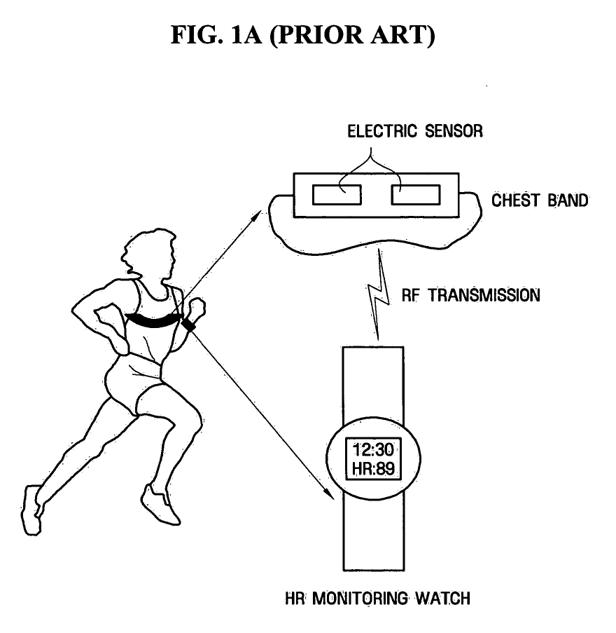

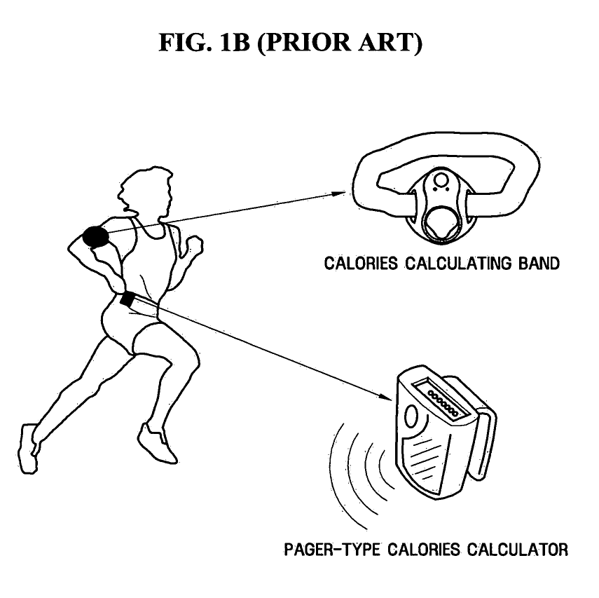

### DETAILED DESCRIPTION OF EXEMPLARY EMBODIMENTS

Advantages and features of the present invention and methods of accomplishing the same may be understood more readily by reference to the following detailed description of the exemplary embodiments and the accompanying drawings. The present invention may, however, be embodied in many different forms and should not be construed as being limited to the embodiments set forth herein. Rather, these embodiments are provided so that this disclosure will be thorough and complete and will fully convey the concept of the invention to those skilled in the art. Like reference numerals refer to like elements throughout the specification.

Hereinafter, exemplary embodiments of the present invention will be described in more detail with reference to the accompanying drawings.

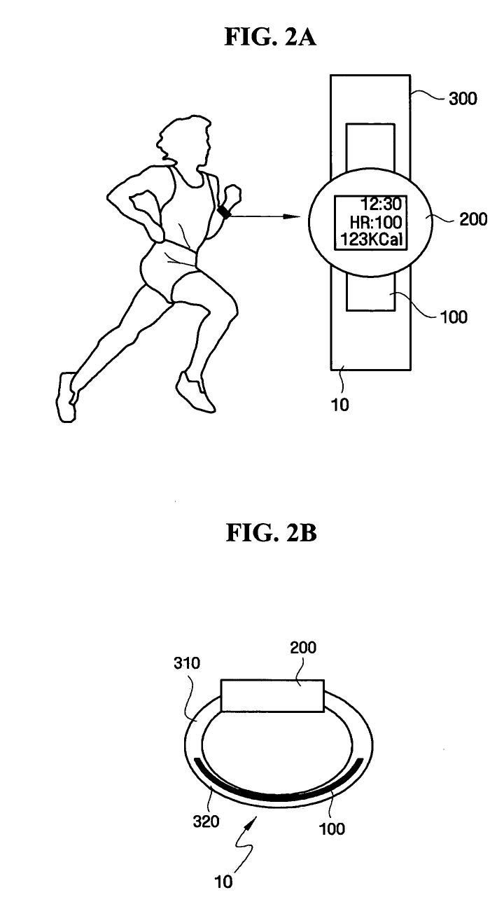

FIGS. 2A and 2B illustrate a construction of an apparatus for measuring the quantity of exercise using a film-type pressure sensor according to an exemplary embodiment of the present invention. FIG. 2A is a top plan view of an exercise quantity measuring apparatus 10, and FIG. 2B is a side view of the exercise quantity measuring apparatus 10.

As illustrated in FIG. 2A, the exercise quantity measuring apparatus 10 comprises a film-type pressure sensor 100, a sensor body 200, and a band 300.

The film-type pressure sensor 100 senses pulse pressure of a user's body. That is, this sensor 100 measures pressure changes in the user's wrist (a user's pulse).

The sensor body 200 calculates a user's HR and the calories burned during exercise based on the measured pulse, and displays the HR and the burned calories. That is, the user's HR is obtained based on the pressure value of the pulse transmitted from the film-type pressure sensor 100, and the calories burned during exercise are calculated using this HR.

In addition, the sensor body 200 removes noise generated as the user moves and while the HR is being measured, to thereby accurately calculate the calories consumed by the user during exercise.

The band 300 to which the film-type pressure sensor 100 is attached couples the sensor body 200 thereto. The band 300 has a predetermined degree of elasticity and comprises a first band part, one end of which is coupled to a side of the sensor body 200, and a second band part coupled to the other end of the first band part, which protects the film-type pressure sensor.

The first band part is coupled to the sensor body 200, which is so elastic that it can adapt to the user's wrist size. The second band part has no elasticity and surrounds the film-type pressure sensor 100, thereby protecting the film-type pressure sensor 100.

The band 300 further comprises a projection positioned on one side of the second band part to enhance the close contact with the user's skin, and a wire to transmit the pulse measured by the film-type pressure sensor 100 to the sensor body 200.

As illustrated in FIG. 2B, the exercise quantity measuring apparatus 10 connects one end of the first band part 310 to one end of the sensor body 200, and the other end of the first band part 310 to one end of the second band part 320. The film-type pressure sensor 100 is attached to the second band part 320 and is used to measure the pulse pressure.

When the user exercises while wearing the wrist watch-type exercise meter having the film-type pressure sensor 100, the user's HR and calories burned can be easily and conveniently obtained.

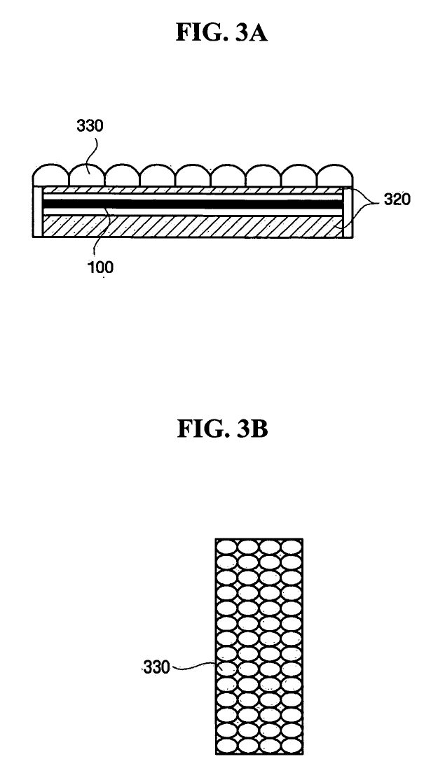

FIGS. 3A and 3B illustrate a construction of a film-type pressure sensor and a band in the exercise quantity measuring apparatus using a film-type pressure sensor according to an exemplary embodiment of the present invention. FIG. 3A is a side sectional view of the film-type pressure sensor 200 and the band 300, and FIG. 3B is a bottom plan view of the band 300.

As illustrated in FIG. 3A, the film-type pressure sensor 100 is surrounded by the second band part 320, which is non-elastic. On one side of the non-elastic second band part 320 (that is, the portion contacting a user's wrist) there is at least one projection 330 to enhance the close contact with the user's skin.

The film-type pressure sensor 100 senses and transmits the pressure changes that occur in a normal pulse. The second band part 320 is used to protect the film-type pressure sensor.

The projection(s) 330 attached to one side of the non-elastic second band part 320 can be a kind of rigid support, which help to more accurately measure the user's pulse.

Generally, the human wrist has an uneven surface because of blood vessels, skin and muscles. For a more precise measurement of the pressure on this uneven surface, the sensor should closely contact the skin. For this purpose, the projection(s) 330, which can be a rigid support, are mounted on one side of the non-elastic second band part 320, enhancing the close contact with the user's skin, and allowing a more precise measurement of the pulse.

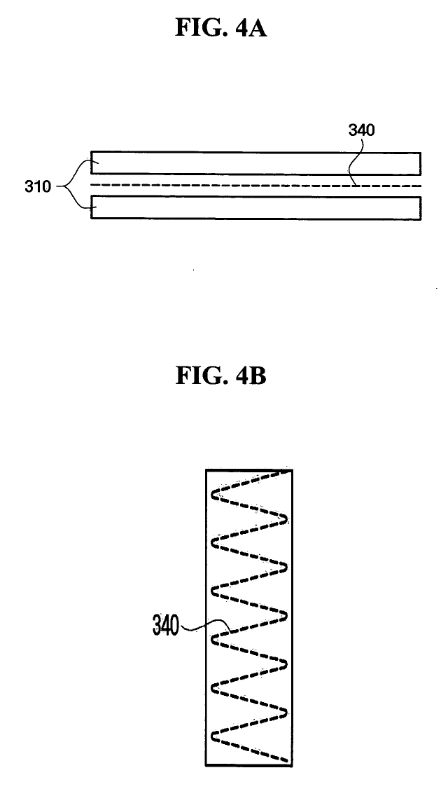

FIGS. 4A and 4B illustrate a construction of the band of the exercise quantity measuring apparatus using a film-type pressure sensor according to an exemplary embodiment of the present invention. FIG. 4A is a side sectional view of the band 300, and FIG. 4B is a top plan section view of the band 300.

As illustrated in FIG. 4A, a wire 340 to transmit the pulse pressure value measured by the film-type pressure sensor 200 is positioned between the elastic first band parts 310. The elastic first band part 310 is used to allow the band of the exercise quantity measuring apparatus 10 to be extended according to the size of the user's wrist.

As illustrated in FIG. 4B, the wire 340 is provided in a wave-form, and not in a linear form. A wave-shaped wire prevents the wire from breaking down due to tension applied thereto when the first band part 310 extends to fit the user's wrist. The wave-shaped wire spreads according to the length of the extended first band part 310.

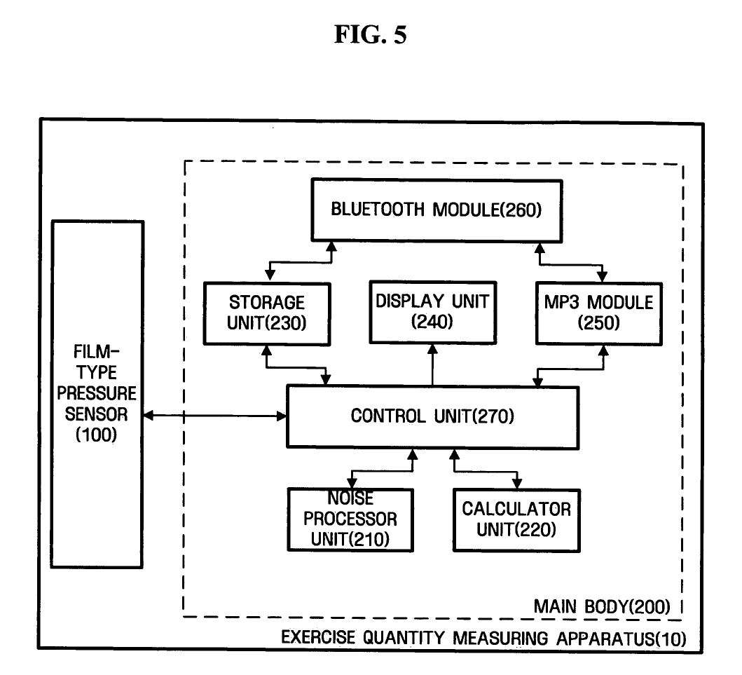

FIG. 5 is a block diagram illustrating an internal construction of the exercise quantity measuring apparatus using the film-type pressure sensor according to an exemplary embodiment of the present invention.

The exercise quantity measuring apparatus comprises a film-type pressure sensor 100 and a sensor body 200. The sensor body comprises a noise processor unit 210, a calculator unit 220, a storage unit 230, a display unit 240, an MP3 module 250, a Bluetooth module 260, and a control unit 270.

The film-type pressure sensor 100 measures the pressure changes caused by the user's pulse. It senses pulse pressure through a pressure sensor shaped like a film.

The noise processor unit 210 removes noise generated when the user moves. It processes the noise generated due to external factors such as the user's movement when the user's pulse is measured using the film-type pressure sensor 100.

For example, the signals transmitted from the film-type pressure sensor 100 contain noise generated by the user's movement. Noise reduction is required to remove it, and this is accomplished through an adaptive filtering method.

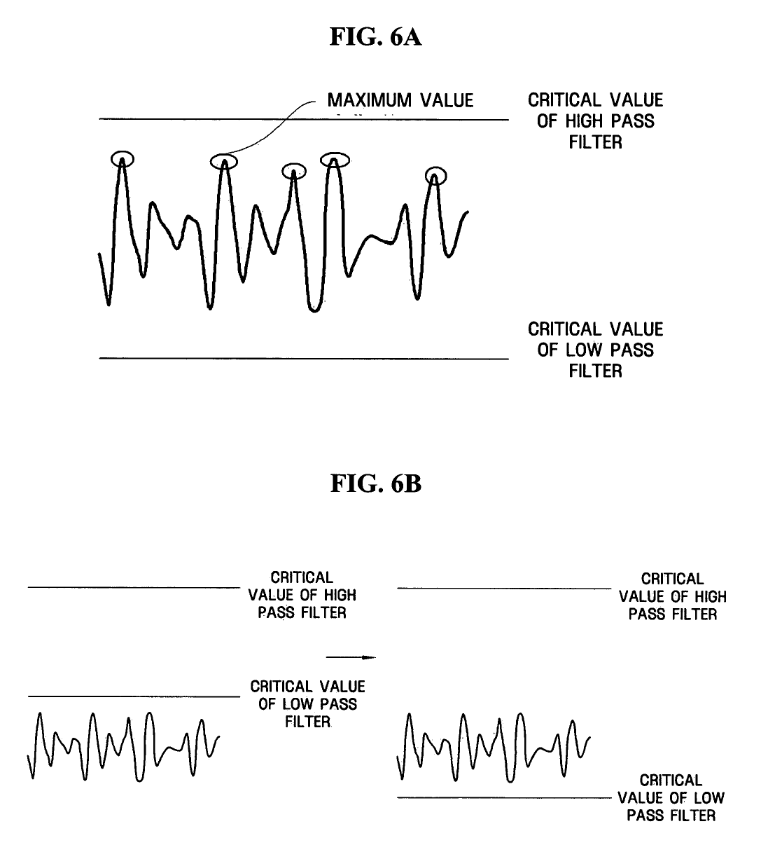

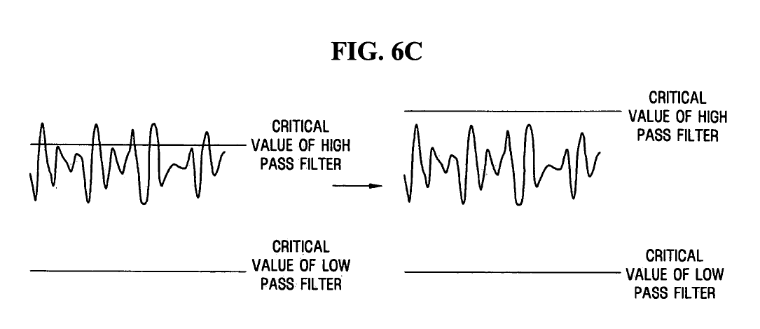

Removal of noise using an adaptive filter will be described with reference to FIGS. 6A, 6B and 6C.

FIGS. 6A, 6B and 6C are graphs drawn using the values transmitted from the film-type pressure sensor 100, which show repetition. The pressure at the peak of the pulse is higher than at other times in the pulse. Secondary peaks occur between the maximum values. The maximum value is the highest value repeated within a predetermined period of time, and this value is repeated in sync with the user's heartbeat.

The heart is contracted at the peak of the pulse. The time between two adjacent upper peaks indicates the time taken for the heart to beat once.

As illustrated in FIG. 6A, the signals inputted as measured from the film-type pressure sensor 100 produce a graph with a characteristic form, and on this graph are lines indicating critical values.

When a maximum value of the graph is lower or higher than a critical value of the low pass filter or the high pass filter, the signals are perceived as noise generated due to the user's movement or an external shock.

Among n maximum values (e.g., n=4) within a predetermined period of time, where only one value is smaller than or larger than the critical value of the low pass filter or the high pass filter, this value is regarded as noise generated due to the user's movement or an external shock, and they are not considered when calculating the user's HR.

However, when all n values are smaller or larger than the critical value of the low pass filter or the high pass filter, they are not determined to be noise but the pulse pressure of the user. Additionally, the critical values of the low pass filter and/or the high pass filter are continuously modified to be the mean of the n maximum values recently input.

As illustrated in FIG. 6B, pulse pressure values transmitted from the film-type pressure sensor 100 are received, and when the received pressure value is a maximum, it is checked whether the recent n maximum values (for example, n=3) are all smaller than the critical value of the low pass filter.

As a result of the checking, when the received maximum values are all smaller than the critical value of the low pass filter, the critical value of the low pass filter is modified to be the mean of the recent three maximum values.

As illustrated in FIG. 6C, it is checked whether the recent n maximum values (for example, n=3) are all larger than the critical value of the high pass filter.

As a result of the checking, when the received maximum values are all larger than the critical value of the high pass filter, the critical value of the high pass filter is modified to be the mean of the recent three maximum values.

Noise-free pressure data can be obtained in this manner.

The calculator unit 220 calculates the user's HR based on the pulse pressure values measured by the film-type pressure sensor 100, and it calculates the calories burned during exercise based on this HR.

The calculator unit 220 comprises a HR calculating module to calculate a HR based on the pulse pressure values, an oxygen quantity calculating module to calculate the oxygen quantity consumed during exercise based on the calculated HR, and a calorie calculating module to calculate the calories consumed during exercise based on the calculated HR and oxygen quantity (all of which are not shown).

Expressions to calculate calories and the HR will now be discussed.

While a user is exercising, energy is consumed. The process of consuming energy is a kind of metabolic oxidization, and oxygen is required in the oxidization.

Thus, consumption of calories while the user is exercising implies that oxygen is consumed. As the calories consumed per unit time and unit weight increase, the quantity of oxygen required per unit time and unit weight increases accordingly.

The expression to obtain the calories E consumed per unit time and unit weight is:
E=aO+b (1)
where O refers to the quantity of oxygen consumed, and a and b are predetermined coefficients defined according to the user information and the kind of exercise. Here, the coefficients a and b are not the same for all people and all kinds of exercises, but they vary according to the gender and weight of the user, and the kind of exercise. Thus, coefficients a and b should be determined according to information regarding the user and the kind of exercise.

More oxygen is supplied when a person breathes fast. If more energy than usual is consumed because of exercise, the number of breaths per minute increases.

The HR per minute and the quantity of oxygen consumed have a linear relationship:
HR=cO+d (2)
where O refers to the quantity of oxygen consumed, and c and d are predetermined coefficients defined according to the user information and the kind of exercise. Here, coefficients c and d are not the same for all persons and all kinds of exercises, but they vary according to the gender and weight of the user, and the kind of exercise. Thus, coefficients c and d should be decided in a proper manner according to information of the user and the kind of exercise

The storage unit 230 stores user information and data about the calories consumed. The user information includes the gender, weight and height of the user. The user information is used by the calculator unit 220 in calculating the consumed calories.

The display unit 240 displays the user's HR and the consumed calories calculated by the calculator unit 220. The display unit 240 also displays the current time, the user's type of exercise, and health-related services.

That is, the display unit 240 provides the user with health and exercise information, by displaying current exercise information, consumed calories, and other information. Here, health and exercise-related services are provided through the Bluetooth module 260.

The Bluetooth module 260 receives user information or music files transmitted from external devices. The external devices may be a personal computer (PC), a notebook computer, a personal digital assistant (PDA), and others; all of which can process Bluetooth signals.

The exercise quantity measuring apparatus 10 not only provides the calories consumed during exercise but it can also be used as a means to receive health-related services such as body care information.

To receive proper services, the user has to transmit the consumed calories to an external device. When user information changes, for example, when the peak HR or the user's weight changes, the new information has to be transmitted to the exercise quantity measuring apparatus 10 to thereby update the user information. The Bluetooth module is used for the transmission and reception of data.

The MP3 module 250 plays music files transmitted via the Bluetooth module 260. The music files transmitted from an external device are received by the Bluetooth module 260, and the transmitted music files are stored in the MP3 module 250. The user can listen to music from the MP3 module 250 through a wireless headset.

Accordingly, the user can listen to music while exercising. By using the wireless headset, the wire entanglement problem that occurs when the user exercises is avoided.

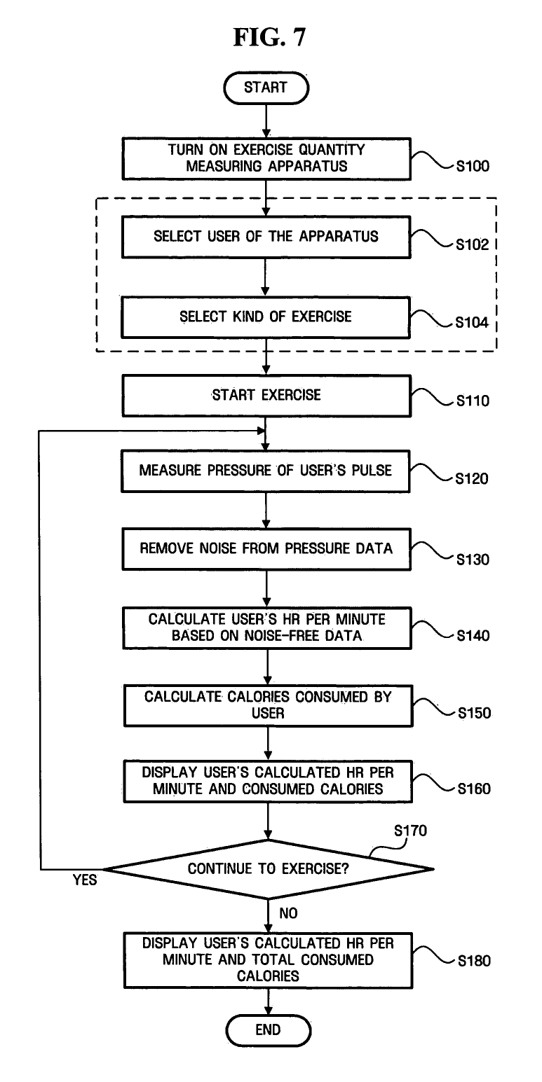

FIG. 7 is a flow chart illustrating an exercise quantity measurement method through a film-type pressure sensor according to an exemplary embodiment of the present invention.

A user first wears the exercise quantity measuring apparatus 10 on their wrist, and turns on the exercise quantity measuring apparatus 10 (S100) before starting an exercise (S110).

When the exercise quantity measuring apparatus 10 is used by a plurality of users, a user who wishes to exercise is selected from a list of the users in the exercise quantity measuring apparatus 10, after turning on the apparatus (S102).

The user selects a type of exercise (S104). Selection of the kind of exercise is necessary because the factors (a, b, c and d) used to calculate the calories burned during exercise change according to the kind of exercise. However, the user may choose not to select the type of exercise.

When the user starts exercising, the pulse pressure measured by the film-type pressure sensor 100 is transmitted to the control unit 270 (S120).

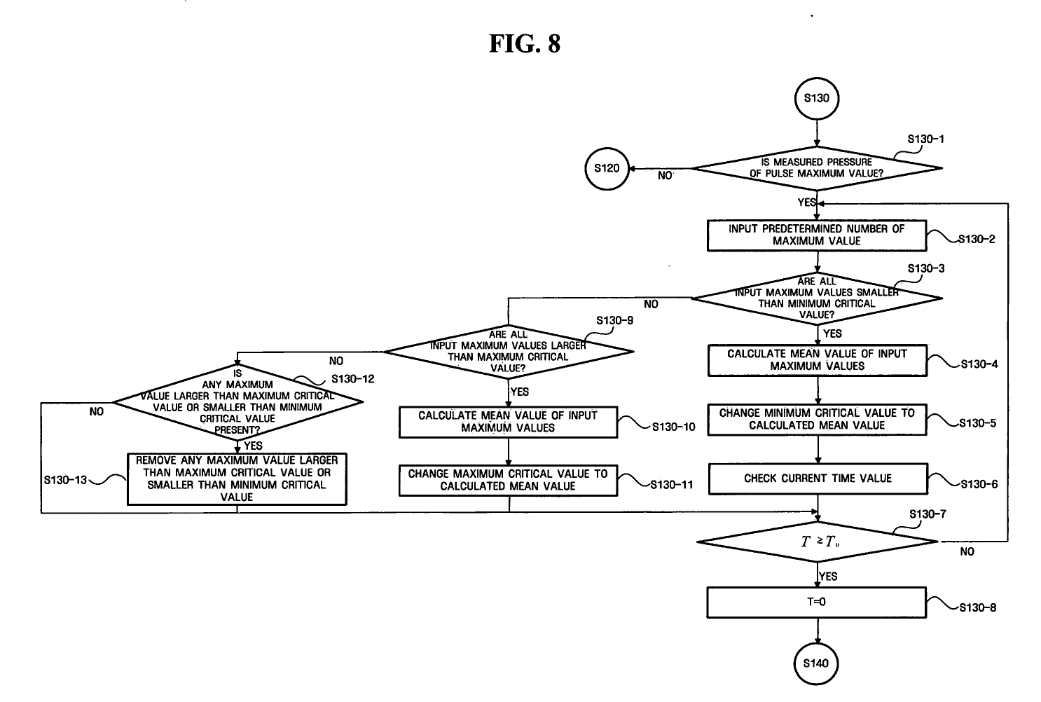

The control unit 270 transmits the received pressure data to the noise processor unit 210, and the noise processor unit 210 removes noise from the transmitted pressure data (S130). The noise processor unit 210 removes noise generated due to external factors such as the user's movement; this removal is performed so as to accurately calculate the user's HR. Removal of the noise signals will be described in detail with reference to FIG. 8.

The calculator unit 220 calculates the user's HR (S140) based on the noise-free pulse pressure using:
HR=60/T (3)
where T refers to the time taken for one heartbeat, which can be known from the pulse pressure graph.

To calculate the calories consumed during exercise using the calculated HR S150, the quantity of oxygen consumed must first be calculated.

To calculate the quantity of oxygen, Expression 2 is used (described above in connection with FIG. 5).

The HR value calculated using Expression 2, and the coefficient values c and d, defined according to the user information and the kind of exercise, are substituted so as to calculate the quantity of oxygen.

The calories consumed during exercise are calculated using the calculated quantity of oxygen (S150).

Expression 1 described above is used to calculate the calories consumed during exercise.

The quantity of oxygen calculated using Expression 2, and the coefficient values a and b, defined according to the user information and the kind of exercise, are substituted into Expression 1 to calculate the consumed calories E.

The calculator unit 220 transmits the calculated HR and the consumed calories to the control unit 270, and the control unit 270 stores the transmitted data values in the storage unit 230 and then displays the user's HR and the consumed calories (S160) in the display unit 240.

Accordingly, the user can know the calories burned while exercising.

Operations S120 to S160 are repeated while the user exercises (S170). When the user terminates exercise, the calculator unit 220 calculates the calories consumed during exercise based on the total exercise time and the consumed calories, and transmits the result to the control unit 270.

The total calories consumed by the user can be obtained by the following expression.
Total calories=E×T (4)

The control unit 270 stores the transmitted data values in the storage unit 230, and displays the user's HR and the consumed total calories through the display unit 240 (S180).

Since the user can be immediately supplied with the user's HR and calories consumed during exercise, the user can directly know how many calories have been consumed during exercise. The user can also monitor the HR while exercising, thereby avoiding physical discomfort.

Through the Bluetooth module 260 of the exercise quantity measuring apparatus 10, the user can be supplied with information about the calories consumed over the course of a day, and can be supplied with health information from an external device.

The user can also input and update user information through the external device, and transmit music files to the MP3 module 250 of the exercise quantity measuring apparatus 10.

The Bluetooth module 260 transmits audio (based on music files supplied by the MP3 module 250) to the wireless headset. Accordingly, exercise is a more pleasant experience.

FIG. 8 is a flow chart of a method to remove noise from the pulse pressure data in the exercise quantity measuring method according to an exemplary embodiment of the present invention.

When the pulse pressure is transmitted to the noise processor unit 210, the noise processor unit 210 determines whether the transmitted pressure is a maximum (S130-1).

When it is determined that a maximum value has been transmitted, the noise processor unit 210 checks whether the predetermined number (e.g., n) of maximum values have been input within an arbitrary time period (S130-2).

Then, the noise processor unit 210 determines whether the input n maximum values are all smaller than the critical value of the low pass filter (S130-3).

When it is determined that the inputted maximum values are all smaller than the critical value of the low pass filter, the noise processor unit 210 calculates the mean of the input maximum values and changes the critical value of the low pass filter to this mean (S130-4) and (S130-5).

Then, the noise processor unit 210 checks the current time and compares the set time value with the current time (S130-6) and (S130-7). As a result of the comparison, when the current time T is larger than To (Time out), the noise processor unit 210 changes the current time to 0, and performs Operation S140.

When the current time T is smaller than To, the noise processor unit 210 performs Operations S130-2 to S130-7 until the set time becomes To.

As a result, when the input maximum values are all larger than the critical value of the set high pass filter (S130-9), the noise processor unit 210 calculates the mean of the input maximum values (S130-10), and changes the critical value of the high pass filter to the calculated mean value (S130-11).

As a result of the checking, when there is present any maximum value larger than the critical value of the high pass filter or smaller than that of the low pass filter (S130-12), this maximum value is removed (S130-13), and then Operation S130-7 is performed.

As described above, the exercise quantity measuring apparatus and method according to the present invention produce at least one of the following effects.

First, the user will not experience physical discomfort since the user is exercising while wearing a wrist-type exercise quantity measuring apparatus having a film-type pressure sensor. In addition, the user can immediately know the calories consumed during exercise through the exercise quantity measuring apparatus.

Second, the user can determine the exercise quantity for one day since the user can obtain information regarding the calories consumed during one day through an external device and the exercise quantity measuring apparatus. Further, since the calories consumed during exercise are immediately ascertained, the efficiency of exercise can be enhanced.

Third, the user can exercise without bearing a physical burden since the user can check his or her HR while exercising.

Fourth, the user can listen to music without hindrance while exercising by using the exercise quantity measuring apparatus having a Bluetooth module and a wireless headset, thereby making the exercise more pleasant.

Those of ordinary skill in the art can understand that various replacements, modifications and changes in the form and details may be made without departing from the spirit and scope of the present invention as defined by the following claims. Therefore, it is to be appreciated that the above described exemplary embodiments are for purposes of illustration only and not to be construed as a limitation of the invention.

What is claimed is:

1. An apparatus for measuring exercise quantity, the apparatus comprising:
a film-type pressure sensor which senses a pulse pressure of a body of a user;
a main body which calculates and displays a heart rate (HR) of the user and calories consumed by the user based on the HR; and
a band that is coupled to the main body and to which the film-type pressure sensor is attached.

2. The apparatus of claim 1, wherein the band comprises:
a first band part having a degree of elasticity, one end of which is coupled to a side of the main body;
a second band part being coupled to the other side of the first band part, to thereby protect the film-type pressure sensor;
at least one projection positioned on one side of the second band part, to enhance sensing of said pulse pressure; and
a wire which transmits the pulse pressure signal produced by the film-type pressure sensor to the main body.

3. The apparatus of claim 2, wherein the wire is positioned within the first band part.

4. The apparatus of claim 3, wherein if the first band part is in a non-extended state the wire is configured in a wave form such that the wire has a length longer than the first band part.

5. The apparatus of claim 2, wherein the second band part is non-elastic.

6. The apparatus of claim 2, wherein the film-type pressure sensor is surrounded by the second band part.

7. The apparatus of claim 2, wherein the wire is surrounded by the first band part.

8. The apparatus of claim 1, wherein the main body comprises:
a noise processor unit which removes noise generated as the user moves;
a calculator unit which calculates the HR based on the measured pulse pressure, and calculates the calories consumed during exercise based on the HR; and
a display unit which displays the HR and the consumed calories.

9. The apparatus of claim 8, wherein the main body further comprises:
a storage unit which stores the consumed calories and user information;
a Bluetooth module which receives the user information or music files from an external device; and
an MP3 module which plays the music files transmitted via the Bluetooth module.

10. The apparatus of claim 1, wherein the HR is a number of beats per minute.

11. A method for measuring exercise quantity using a film-type pressure sensor, comprising:
measuring the pulse pressure of a user who is exercising;
removing noise from the pulse pressure signal to provide a noise free pulse pressure;
calculating a heart rate (HR) of the user, based on the noise-free pulse pressure;
calculating the calories consumed by the user based on the HR; and
displaying the calories consumed and the HR.

12. The method of claim 11, wherein the pulse pressure is measured using the film-type pressure sensor.

13. The method of claim 11, wherein the removing the noise from the pulse pressure signal comprises:
checking whether a predetermined number of maximum values are input within a set time period;
determining whether the input maximum values are all smaller than a set critical value of a low pass filter and whether the input maximum values are larger than a set critical value of a high pass filter, when the predetermined number of maximum values are input;
calculating the mean of the input maximum values, if it is determined that the input maximum values are all smaller than the set critical value of the low pass filter, and changing the critical value of the low pass filter to the mean;
calculating the mean of the input maximum values, if it is determined that the input maximum values are all larger than the set critical value of the high pass filter, and changing the critical value of the high pass filter to the mean;
determining whether there is present any maximum value larger than the critical value of the high pass filter or smaller than that of the low pass filter; and
if it is determined that there is present a maximum value larger than the critical value of the high pass filter or smaller than that of the low pass filter, removing the maximum value present.

14. The method of claim 11, further comprising selecting a user profile if a plurality of users use the exercise quantity measuring apparatus.

15. The method of claim 11, further comprising selecting a type of exercise that the user will engage in.

16. The method of claim 11, further comprising measuring the pulse pressure of the user through at least one projection on a band coupled to a wrist of the user.
